(19) United States
(12) Patent Application Publication
(10) Pub. No.: US 2009/0254360 A1
(43) Pub. Date: Oct. 8, 2009

(54) Systems and Methods For Healthcare Asset Allocation

(75) Inventor: Suresh K. Choubey, Delafield, WI (US)

Correspondence Address: MCANDREWS HELD & MALLOY, LTD, 500 WEST MADISON STREET, SUITE 3400, CHICAGO, IL, 60661, US

(73) Assignee: GENERAL ELECTRIC COMPANY, Schenectady, NY (US)

(21) Appl. No.: 12/061,452

(22) Filed: Apr. 2, 2008

(51) Int. Cl. G06Q 10/00

(52) U.S. Cl. 705/2

(57) ABSTRACT

Certain embodiments of the present invention provide a healthcare asset allocation method including receiving a healthcare asset request, creating a plurality of potential solutions using healthcare asset data, and determining a healthcare asset allocation by applying a genetic algorithm to the plurality of potential solutions using a plurality of genetic algorithm parameters and the healthcare asset request. The healthcare asset data includes data related to assets in a healthcare enterprise.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to healthcare assets. More particularly, the present invention relates to systems and methods for healthcare asset allocation. Asset allocation may include initially allocating assets and/or reallocating assets.

Healthcare assets in a healthcare enterprise, such as a single or group of hospitals, other healthcare providers, and/or hospital systems which multiple satellite sites, may consist of IV pumps, wheel chairs, patient beds, imaging equipment, medicine, blood, surgical tools, and other biomedical or healthcare related equipment and supplies. The healthcare assets may be underutilized because each hospital or provider department in the enterprise may not have the right amount and/or type of assets at a certain time. The need for certain types of assets may fluctuate in time and from department to department. Purchasing more assets than are needed for the enterprise as a whole may be too expensive. Further, the healthcare enterprise may have multiple departments in multiple facilities. Surplus assets in one department of the enterprise may be used to supplement a need for assets in another department of the enterprise, which may help to improve the overall enterprise level asset utilization.

However, a department needing a certain type of asset may not know that another department in the enterprise has a surplus of that asset type. During certain times of the day, month, and/or year, one department may need more or less of a certain type of asset. Purchasing and storing extra assets can be expensive and time consuming. Similarly, renting extra assets can pose the same problems. These approaches may lead to unwarranted purchases of assets at an enterprise and a department level. Furthermore, asset acquisition requests may be generated on a department by department basis, as opposed to using an enterprise-wide basis. Hence, any additional or unused assets in one department may not satisfy the need in another department.

### BRIEF SUMMARY OF THE INVENTION

Certain embodiments of the present invention provide a healthcare asset allocation method including receiving a healthcare asset request, creating a plurality of potential solutions using healthcare asset data, and determining a healthcare asset allocation by applying a genetic algorithm to the plurality of potential solutions using a plurality of genetic algorithm parameters and the healthcare asset request. The healthcare asset data includes data related to assets in a healthcare enterprise.

Certain embodiments of the present invention provide a healthcare asset allocation system including a healthcare asset database component adapted to store healthcare asset data, and a healthcare asset allocation component adapted to determine a healthcare asset allocation using a genetic algorithm, the stored healthcare asset data, and a plurality of genetic algorithm parameters.

Certain embodiments of the present invention provide a computer-readable medium including a set of instructions for execution on a computer, the set of instructions including a data retrieval routine, a user interface routine, and an asset allocation routine. The data retrieval routine is configured to retrieve healthcare asset data. The user interface routine is configured to receive a healthcare asset request. The asset allocation routine is configured to determine a healthcare asset allocation using a genetic algorithm, a plurality of genetic algorithm parameters, the healthcare asset request, and the healthcare asset data.

### BRIEF DESCRIPTION OF SEVERAL VIEWS OF THE DRAWINGS

FIG. 1 illustrates a flow diagram for a method for healthcare asset allocation in accordance with an embodiment of the present invention.

FIG. 2 illustrates a flow diagram for a method for applying a genetic algorithm to a plurality of potential solutions to determine a healthcare asset allocation in accordance with an embodiment of the present invention.

FIG. 3 illustrates a flow diagram for a method for healthcare asset allocation in accordance with an embodiment of the present invention.

FIG. 4 illustrates a plurality of potential solutions in accordance with an embodiment of the present invention.

FIG. 5 illustrates a healthcare asset allocation system in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentalities shown in the attached drawings.

### DETAILED DESCRIPTION OF THE INVENTION

Certain embodiments of the present invention provide a genetic-algorithm based approach for healthcare asset allocation and/or reallocation from departments with surplus assets to departments needing those assets. Certain embodiments may seek to minimize the total cost of moving, purchasing, renting, and/or storing assets, or maximize the enterprise-wide asset allocation while limiting a race condition. The race condition may occur when a donor department gives away its assets to a requesting department based on its current availability and then needs the same type of assets in a couple hours; thus triggering a request for the same type of assets.

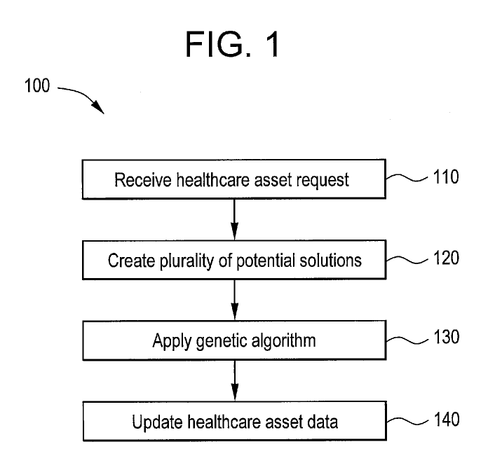

FIG. 1 illustrates a flow diagram for a method 100 for healthcare asset allocation in accordance with an embodiment of the present invention. The method 100 includes the following steps, which will be described below in more detail. At step 110, a healthcare asset request is received. At step 120, a plurality of potential solutions is created using healthcare asset data. At step 130, a genetic algorithm is applied to the plurality of potential solutions using a plurality of genetic algorithm parameters and the healthcare asset request to determine a healthcare asset allocation. At step 140, the healthcare asset data is updated using the healthcare asset allocation. The method 100 is described with reference to elements of systems described below, but it should be understood that other implementations are possible.

At the step 110, the healthcare asset request is received. The request may be generated by a hospital or enterprise employee, or automatically generated by a healthcare asset allocation system, such as the system 500 described below. The system may automatically generate a healthcare asset request based on healthcare asset data available to the system. For example, the system may predict that more healthcare assets of a certain type will be needed based on the number of operations scheduled in the operating room for a certain day. A healthcare asset may be an IV pump, a wheel chair, a patient bed, an imaging machine, a surgical tool, a medication, blood, saline, or other biomedical or healthcare equipment or supplies. These are examples of different types of healthcare assets. For example, the number of asset types may be less than 10,000. The healthcare asset request is a request for a certain amount of a type of healthcare asset. The healthcare asset request may include the type of healthcare asset requested, the amount of the type requested, and the time of the request. For example, the healthcare asset request may be a request for 8 IV pumps. The healthcare asset request may be submitted by a healthcare department in a healthcare enterprise, for example. The healthcare enterprise may have multiple departments in the same or multiple different facilities. The healthcare asset request may be submitted to a healthcare asset allocation component through a user interface component, for example.

In certain embodiments, the user interface component may be accessed through a local network internal to the enterprise. In certain embodiments, the user interface component may be accessed through a web service bridge. The web service bridge may be any combination of hardware and/or software adapted to provide access to the user interface component over the internet. In certain embodiments, the user interface component may display a web page provided by the web service bridge. The web service bridge would be adapted to process input and output. The user may use any computer or network device to interact with the web page in order to send a request to and receive results from the user interface component. The web service bridge enables the integration of and communication between various systems and diversified platforms.

At the step 120, the plurality of potential solutions is created using the healthcare asset data. The healthcare asset data may be stored in a healthcare asset database component, which is further described below. The step 120 may further include retrieving the healthcare asset data from the healthcare asset database component.

The plurality of potential solutions may be created using one or more genetic algorithm parameters. The plurality of genetic algorithm parameters may include an approach type, a crossover rate, a crossover type, a mutation rate, a cost function, a population size, a selection method, a convergence criterion, and/or an end condition. In certain embodiments, the crossover rate may be 95 percent, and the mutation rate may be 2 percent, for example. In certain embodiments, the selection method may be a roulette wheel selection method, for example.

The population size is the number of potential solutions. For example, the population size may be 30.

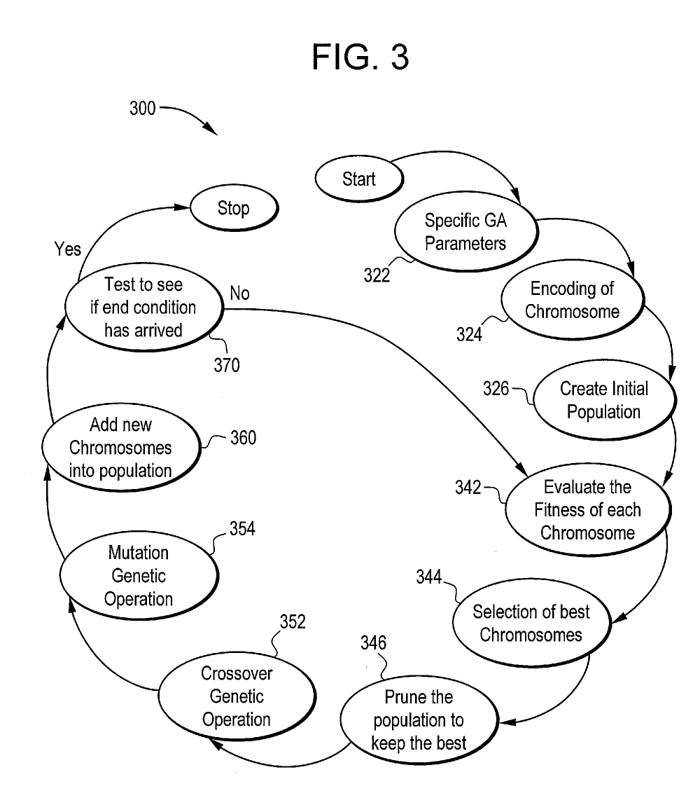

As described below in reference to FIG. 3, the step 120 may also be considered part of the genetic algorithm. The genetic algorithm may be implemented in a variety of ways. Although examples of how to create a plurality of potential solutions and apply the genetic algorithm to the potential solutions are provided below, the examples are not meant to limit any embodiment of the invention. Genetic algorithms generally are known.

Genetic algorithms may provide computational models mimicking natural evolution (survival of the fittest and change in solution space using natural production techniques such as crossover and mutation) to solve complex optimization problems. Genetic algorithms belong to a Guided Random Search Techniques Class, which uses enumerative techniques and additional information to guide the search. Another method of this type, called simulated annealing, uses a thermodynamic evolution process to search minimum energy states.

A genetic algorithm may define the population of the search space and a set of well defined operations from the natural evolution domain. The operations may help in the selection of the set of best solutions that has right to multiply—similar to a biological system. In terms of asset allocation/reallocation, each solution in the solution space may become an individual member of the search space (also called population). Each member may be encoded as a string of numbers. The genetic algorithm may then manipulate the most promising individual in the search space for an improved solution. The genetic algorithm may begin with a chromosome population containing a set of possible solutions.

The enterprise may include one or more facilities. Each facility may include one or more departments. By way of an example, a healthcare enterprise may include two facilities: Facility 1 and Facility 2. Facility 1 may include a surgery department (surgery1), an emergency department (emergency1), a pediatrics department (pediatrics1), an outpatient department (outpatient1), and a cardiology department (cardiology1). Facility 2 may include a surgery department (surgery2), an emergency department (emergency2), a pediatrics department (pediatrics2), an obstetrics department (obstetrics2), and a neurology department (neurology2).

The healthcare asset data may include information concerning the healthcare assets, such as predicted, present, and/or past amounts of the healthcare assets, amounts of excess healthcare assets, patient flow, hospital occupancy, patient workflow, rates of use of the healthcare assets, diagnostic related groups (DRGs) within the hospital, and/or other information related to hospital, commercial, and/or financial data. For example, a patient may come into the hospital complaining of chest pain. The patient may flow through a predictable series of departments, tests, and/or examinations related to the same DRG. Thus, the needed assets may also be predictable.

As another example, the healthcare asset data for each department's supply of extra IV pumps may be: surgery1=2, emergency1=4, pediatrics1=0, outpatient1=3, cardiology1=1, surgery2=1, emergency2=3, pediatrics2=1, obstetrics2=2, and neurology2=4.

The potential solutions may be created using a variety of methods. For example, each potential solution may be created by randomly ordering the departments in the solution.

The arrangement of the potential solution may vary according to several different factors, such as the number of assets being allocated, the size of the enterprise, the asset allocation profile, a patient entry profile, diagnostic procedures to be performed, known and predicted future asset needs, and the interchangeability of certain assets. For example, the potential solution may list all the departments and provide excess assets from the departments in the order they are listed. In that example, the requesting department's value in the list would be set to 0, as the requesting department needs more assets. The departments may be randomly ordered to create the plurality of potential solutions. For example, one potential solution may be emergency1, outpatient1, surgery2, pediatrics1, obstetrics2, emergency2, pediatrics2, surgery1, neurology2, cardiology1. If the healthcare asset request is that surgery2 needs 8 IV pumps, this solution would provide surgery2 with emergency1's 4 IV pumps, outpatient1's 3 IV pumps, and 1 of obstetrics2's IV pumps. Surgery2 requested the pumps, and thus would not be able to supply them. Pediatrics1 did not have any surplus pumps.

The number of potential solutions may be equal to the population size. If the population size is 30, then 30 potential solutions may be randomly generated.

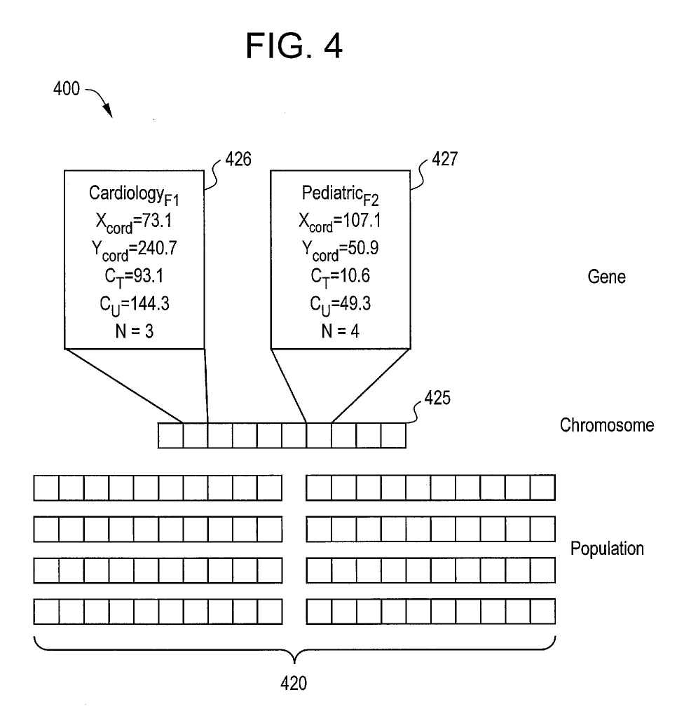

The plurality of potential solutions may be called a population. Each potential solution may also be called a chromosome. The chromosome may have one or more genes which together represent the potential solution. For example, a gene in the above potential sample would be any of the departments listed, such as emergency1, along with the necessary healthcare asset data for the solution, such as the number of excess assets of the type requested in that department. The terms gene, chromosome, and population are further described below in reference to FIG. 4.

In certain embodiments, the step 120 may further include specifying the genetic algorithm parameters. For example, the user interface component may display fields for the user to input values for the genetic algorithm parameters. The fields may include drop-down boxes displaying options for one or more of the genetic algorithm parameters. The method 100 may then use these inputted genetic algorithm parameters in creating the potential solutions and applying the genetic algorithm.

At the step 130, the genetic algorithm is applied to the plurality of potential solutions using the plurality of genetic algorithm parameters and the healthcare asset request to determine the healthcare asset allocation.

The approach type may be a generational approach type or a steady state approach type, for example. In the generational approach type, one or more potential solutions are changed at a time. In the steady state approach type, only one potential solution is changed at a time.

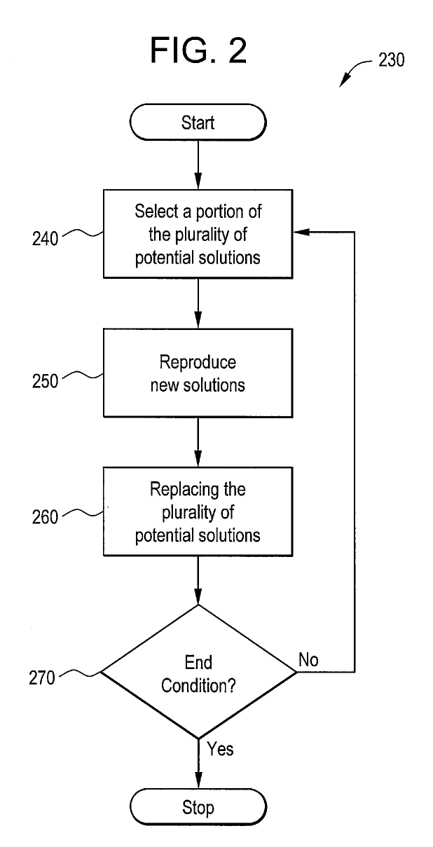

In certain embodiments, the genetic algorithm may comprise the steps of: selecting a portion of the plurality of potential solutions using the cost function; reproducing one or more new solutions using the selected portion; replacing the plurality of potential solutions with the one or more new solutions and the selected portion; and repeating the selecting, reproducing, and replacing steps until the end condition occurs. These steps are described below in reference to FIG. 2.

At the step 140, the healthcare asset data is updated using the healthcare asset allocation. The method 100 may be performed without step 140. If a potential solution is selected, the healthcare asset allocation component may update the healthcare asset data in the healthcare asset database component with the healthcare asset allocation.

Certain embodiments of the present invention may omit one or more of these steps and/or perform the steps in a different order than the order listed. For example, some steps may not be performed in certain embodiments of the present invention. As a further example, certain steps may be performed in a different temporal order, including simultaneously, than listed above.

One or more of the steps of the method 100 may be implemented alone or in combination in hardware, firmware, and/or as a set of instructions in software, for example. Certain embodiments may be provided as a set of instructions residing on a computer-readable medium, such as a memory, hard disk, DVD, or CD, for execution on a general purpose computer or other processing device.

FIG. 2 illustrates a flow diagram for a method 230 for applying a genetic algorithm to a plurality of potential solutions to determine a healthcare asset allocation in accordance with an embodiment of the present invention. The method 230 may use a plurality of genetic algorithm parameters and a healthcare asset request. The method 230 may be similar to the step 130 described above. The genetic algorithm parameters may be similar to the genetic algorithm parameters discussed above in reference to FIG. 1. The healthcare asset request may be similar to the healthcare asset request discussed above in reference to FIG. 1.

The method 230 includes the following steps, which will be described below in more detail. At step 240, a portion of the plurality of potential solutions is selected using a cost function. At step 250, one or more new solutions are reproduced using the selected portion. At step 260, the plurality of potential solutions is replaced with the one or more new solutions and the selected portion. At step 270, if an end condition occurs then the method 230 is completed. If the end condition has not occurred, the steps 240, 250, 260, and 270 are repeated. The method 230 is described with reference to elements of systems described below, but it should be understood that other implementations are possible.

At the step 240, the portion of the plurality of potential solutions is selected using the cost function. The cost function may determine a cost of a potential solution. In certain embodiments, the cost function may include a distance between a donor department and a requesting department, a transfer cost, a utilization cost, and/or a facility transfer cost. For example, the cost function may include the following equation:

Cₖ=Σ_{i=1,n}[(Xᵢ−Xₖ)²+(Yᵢ−Yₖ)²]^{1/2}+C_{T}+C_{U}+C_{Fik}

In certain embodiments, the cost function may include the following equation:

Cₖ=Σ_{i=1,n}([(Xᵢ−Xₖ)²+(Yᵢ−Yₖ)]^{1/2}+C_{T}+C_{U}+C_{Fik})

Where, Cₖ=cost of transferring assets between all the donor departments and department k (requesting department); n=the number of departments in the enterprise; Xᵢ=X coordinates of the donor department i; Xₖ=X coordinates of the requesting department k; Yᵢ=Y coordinates of donor department i; Yₖ=Y coordinates of requesting department k; C_{T}=Cost of transfer for the healthcare asset type; C_{U}=Cost of utilization for the healthcare asset type; and C_{Fik}=Cost of transferring the healthcare asset type from the donor department i's facility to the requesting department k's facility—where the C_{Fik} will be 0 if the donor department and the requesting department are in the same facility. In certain embodiments, C_{Fik} may be a fixed cost as long as the donor department and the requesting department are not in the same facility. The portion of the cost function which reads [(Xᵢ−Xₖ)²+(Yᵢ−Yₖ)²]^{1/2} may constitute the distance between the donor department and the requesting department. In certain embodiments, the X and Y coordinates may correspond to the longitude and latitude of the departments, respectively. In certain embodiments, an enterprise may define its own X and Y coordinates for each department.

In the above example, the potential solution of emergency1, outpatient1, surgery2, pediatrics1, obstetrics2, emergency2, pediatrics2, surgery1, neurology2, cardiology1 may result in a cost function which sums up: (1) the distances between surgery2 and emergency1, outpatient1, and obstetrics2, respectively, as those are the donor departments which would transfer assets; (2) the C_{T} for transferring IV pumps and the C_{U} for using the IV pumps; and (3) the C_{Fik} for each transfer between two facilities—namely between emergency1 and surgery2, and between outpatient1 and surgery2, but not between obstetrics2 and surgery2.

The C_{T} for transferring IV pumps and/or the C_{U} for using the IV pumps may be specific to a donor department. The C_{T} may also include the costs associated with avoiding race conditions, such as the race conditions posed by future utilization of the assets by the donor department.

A department is not a donor department if none of its assets are being transferred according to the potential solution.

The healthcare asset data may further include the values for the X and Y coordinates for each department, and the C_{T}, C_{U}, and C_{Fik} for each healthcare asset type in the enterprise. The healthcare asset data may also be stored in the healthcare asset database component. In certain embodiments, the values for C_{T}, C_{U}, and C_{Fik} in the cost function may not account for the number of the healthcare asset type being sent from the donor department to the requesting department. In those embodiments, the values for C_{T}, C_{U}, and C_{Fik} may be multiplied by Aᵢ (the number of assets being transferred from the donor department i to the requesting department k). Consequently, the cost function may be:

Cₖ=Σ_{i=1,n}([(Xᵢ−Xₖ)²+(Yᵢ−Yₖ)²]^{1/2}+Aᵢ(C_{T}+C_{U}+C_{Fik}))

The step 240 may be performed using the selection method. The cost function may be used to determine the cost of each potential solution. The selection method may be a known selection method, such as a roulette wheel selection method, a tournament selection method, or an elitism selection method, for example.

If the selection method is the roulette wheel selection method, a lower cost increases the potential solution's chance of being selected. By way of an analogy, each potential solution can be given a piece of a pie chart. The total of the pieces for each potential solution in the plurality of potential solutions (i.e., the population) creates one pie chart. The size of each piece is inversely proportional to the cost of the potential solution—if the cost is lower, the piece will be larger. If the piece is larger, the potential solution is more likely to be selected.

If the selection method is the tournament selection method, a sample number of potential solutions are selected at random, and the solution with the lowest cost is selected. The sample number may be 2 for example.

If the selection method is the elitism selection method, the potential solution with the lowest cost is selected and the remainder of the selected portion is selected using another selection method.

In certain embodiments, the plurality of potential solutions is limited to only include the selected portion.

At the step 250, the one or more new solutions are reproduced using the selected portion. In certain embodiments, the reproducing step may include performing a cross-over operation on the selected portion at the crossover rate using the crossover type, and performing a mutation operation on the crossed-over selected portion at the mutation rate to form the one or more new solutions. In certain embodiments, the mutation operation may be performed before the crossover operation.

In certain embodiments, the crossover operation and/or the mutation operation may be performed on the selected portion and/or any new solutions. Each time a crossover operation is performed, two new solutions may be created. Each time a mutation operation is performed, one new solution may be created.

To perform the crossover operation, a first potential solution and a second potential solution are selected. One or more crossover points may be selected randomly in the first potential solution according to the crossover type. Single or multiple point crossovers are known in the art. If the crossover type is a single point crossover, then only one crossover point is selected in the first potential solution. A first new solution and a second new solution may be created from the crossover. Everything before the crossover point on the first potential solution may be copied into the first new solution. Everything after the first crossover point on the first potential solution may be copied into the second new solution. The remainder of the first new solution and the second new solution may be filled in with the second potential solution.

By way of an example, if potential solution A (emergency1, outpatient1, surgery2, pediatrics1, obstetrics2, emergency2, pediatrics2, surgery1, neurology2, cardiology1) is crossed over using a single crossover point with potential solution B (neurology2, cardiology1, outpatient1, surgery2, pediatrics1, pediatrics2, emergency2, obstetrics2, surgery1, emergency1), a crossover point may be randomly selected in potential solution A. If the crossover point is the third position in the potential solution, then the crossover operation creates new solution A (emergency1, outpatient1, surgery2, neurology2, cardiology1, pediatrics1, pediatrics2, emergency2, obstetrics2, surgery1) and new solution B (outpatient1, surgery2, emergency1, pediatrics1, obstetrics2, emergency2, pediatrics2, surgery1, neurology2, cardiology1).

The crossover operation may be performed at the crossover rate. For example, if the crossover rate is 95% and the operation is performed on the selected portion, then 95% of the potential solutions in the selected portion will be crossed over. Restated, the potential solutions will be crossed over 95% of the time. The number of potential solutions crossed over may equal the number of new solutions created.

To perform the mutation operation, a number of genes in a potential solution may be selected to be mutated. For example, two genes may be selected in the potential solution C (emergency1, outpatient1, surgery2, pediatrics1, obstetrics2, emergency2, pediatrics2, surgery1, neurology2, cardiology1). The two genes may be the fourth position and the seventh position, which would make new solution C=emergency1, outpatient1, pediatrics2, pediatrics1, obstetrics2, emergency2, surgery2, surgery1, neurology2, cardiology1. The selected portion and/or the new solutions may be mutated at the mutation rate. For example, if the mutation rate is 2%, then 2% of the potential solutions will be mutated. Restated, the mutation operation will be performed on a potential solution 2% of the time.

At the step 260, the plurality of potential solutions is replaced with the one or more new solutions and the selected portion. The one or more new solutions and the selected portion may be combined to form the plurality of potential solutions. This replaces the plurality of potential solutions from before the step 240. The population size may remain the same if the number of new solutions and the number of potential solutions in the selected portion add up to the population size.

At the step 270, if the end condition occurs then the method 230 is completed. If the end condition has not occurred, the steps 240, 250, 260, and 270 are repeated. The end condition may take into consideration a variety of different factors, such as the cost to rent the requested assets, the cost to purchase and store the requested assets, the number of iterations (generations) the genetic algorithm has run, any convergence criteria, and/or whether a solution exists with a low enough cost using the cost function, for example. To detect the end condition, the step 270 may use the cost function to determine the cost for each of the plurality of potential solutions. For example, the end condition may occur when the number of iterations equals 10. In another example, the end condition may occur if the result of the cost function for a potential solution is less than the cost to rent and/or purchase and store the requested assets. If the cost function is used in the step 270, then the step 240 may use the results of the cost function instead of applying the cost function again.

Certain embodiments of the present invention may omit one or more of these steps and/or perform the steps in a different order than the order listed. For example, some steps may not be performed in certain embodiments of the present invention. As a further example, certain steps may be performed in a different temporal order, including simultaneously, than listed above.

One or more of the steps of the method 230 may be implemented alone or in combination in hardware, firmware, and/or as a set of instructions in software, for example. Certain embodiments may be provided as a set of instructions residing on a computer-readable medium, such as a memory, hard disk, DVD, or CD, for execution on a general purpose computer or other processing device.

FIG. 3 illustrates a flow diagram for a method 300 for healthcare asset allocation in accordance with an embodiment of the present invention. The method 300 includes the following steps, which will be described below in more detail. At step 322, genetic algorithm parameters are specified. At step 324, a plurality of chromosomes is encoded. At step 326, a population of the plurality of chromosomes is created. At step 342, the fitness of each of the plurality of chromosomes is evaluated. At step 344, a portion of the plurality of chromosomes is selected using a cost function. At step 346, the population is pruned to only include the selected portion. At step 352, a crossover operation is performed on the population at a crossover rate using a crossover type. At step 354, a mutation operation is performed on the population at a mutation rate. At step 360, new chromosomes are added to the population. At step 370, if an end condition occurs then the method 300 is completed. If the end condition has not occurred, the steps 342, 344, 346, 352, 354, 360, and 370 are repeated. The method 300 is described with reference to elements of systems described below, but it should be understood that other implementations are possible.

The method 300 may be similar to the method 100 described above. As discussed above, the method 100 may include the method 230. The step 120 may be similar to the step 322, the step 324, and the step 326. The step 130 and/or method 230 may be similar to the steps 342, 344, 346, 352, 354, 360, and 370. The step 240 may be similar to the step 342, the step 344, and the step 346. The step 250 may include the step 352 and the step 354.

At the step 322, the genetic algorithm parameters are specified. The genetic algorithm parameters may be similar to the genetic algorithm parameters discussed above in reference to FIGS. 1 and 2. A user may specify the genetic algorithm parameters using a user interface component. The user interface component may be similar to the user interface component described above in reference to FIG. 1.

At the step 324, the plurality of chromosomes is encoded. Each chromosome may be similar to the potential solutions and/or the chromosomes described above in reference to FIGS. 1 and 2. The plurality of chromosomes may be encoded just as the plurality of potential solutions may be created as discussed above in reference to the step 120 in FIG. 1. The chromosomes may be coded using either binary encoding, permutation encoding, direct value encoding, and tree encoding, for example. For the asset allocation/reallocation, the departments with surplus requested assets may be ranked. This may be similar to a shortest distance problem or traveling sales person problem. Determining the order of the departments to keep the cost at a minimum is an np-hard problem and the time complexity is O(n!), where n is the number of departments. The genetic algorithm may provide much better performance.

At the step 326, the population of the plurality of chromosomes is created. By encoding the plurality of chromosomes in the step 324, the population of the plurality of chromosomes is created. The population may be limited to a population size. The population size may be 30, for example.

At the step 342, the fitness of each of the plurality of chromosomes is evaluated. A cost function may be used to evaluate the fitness of each of the plurality of chromosomes. The cost function may be similar to the cost function described above in reference to the step 240 of FIG. 2. The fitness of each of the plurality of chromosomes may be similar to the cost of each of the plurality of potential solutions as discussed above in reference to the step 240 of FIG. 2.

At the step 344, the portion of the plurality of chromosomes is selected using the cost function. The chromosomes may be selected using a selection method. The selection method may be similar to the selection method discussed above in reference to the step 240 of FIG. 2. The selection method may be a roulette wheel selection method, for example. The selected portion may be an amount smaller than the population size. The selected portion and the new chromosomes/new solutions may add up to the population size. For example, if the population size is 30, the selected portion may have 16 chromosomes/potential solutions and there may be 14 of the new chromosomes/new solutions. The size of the selected portion may be determined by the population size, the crossover rate, and the mutation rate. If the crossover and mutation operations are performed on the selected portion, the size of the selected portion may equal the population size divided by one plus the crossover rate. For example, if the crossover rate is 95%, the mutation rate is 2%, and the population size is 197, the size of the selected portion may be 100. In that example, 95% of the selected portion is crossed over, resulting in 95 new chromosomes, and 2% of the selected portion is mutated, resulting in 2 new chromosomes. Thus the 97 new chromosomes and the 100 chromosomes in the selected portion may be combined to form the 197 chromosomes in the population. Thus, in certain embodiments, the population size remains the same. The genetic operation principles are known in the art.

At the step 346, the population is pruned to only include the selected portion. The population may be limited to only the selected portion.

At the step 352, the crossover operation is performed on the population at the crossover rate using the crossover type. The crossover operation may be similar to the crossover operation described above in reference to the step 250 of FIG. 2.

At the step 354, the mutation operation is performed on the population at the mutation rate. The mutation operation may be similar to the mutation operation described above in reference to the step 250 of FIG. 2.

At the step 360, the new chromosomes are added to the population. The step 360 may be similar to the step 260.

At the step 370, if the end condition occurs then the method 300 is completed. If the end condition has not occurred, the steps 342, 344, 346, 352, 354, 360, and 370 are repeated. The step 370 may be similar to the step 270.

Certain embodiments of the present invention may omit one or more of these steps and/or perform the steps in a different order than the order listed. For example, some steps may not be performed in certain embodiments of the present invention. As a further example, certain steps may be performed in a different temporal order, including simultaneously, than listed above.

One or more of the steps of the method 300 may be implemented alone or in combination in hardware, firmware, and/or as a set of instructions in software, for example. Certain embodiments may be provided as a set of instructions residing on a computer-readable medium, such as a memory, hard disk, DVD, or CD, for execution on a general purpose computer or other processing device.

FIG. 4 illustrates a plurality of potential solutions 400 in accordance with in accordance with an embodiment of the present invention. The plurality of potential solutions 400 includes a population 420, a chromosome 425, a gene 426, and a gene 427.

The population 420 may be similar to the population and the plurality of potential solutions described above in reference to FIGS. 1, 2, and 3. As described above, the population 420 may consist of a plurality of chromosomes. Each of the chromosomes may be similar to the potential solutions described above in reference to FIGS. 1, 2, and 3. The chromosome 425 may represent a potential solution for a requested asset type. The gene 426 and the gene 427 may be similar to the genes described above in reference to FIG. 1. For example, the gene 426 represents a cardiology department at Facility 1. The department is located at an X coordinate 73.1 and a Y coordinate 240.7. There is a transfer cost of 93.1 and a utilization cost of 144.3 for the requested asset type. The department has an excess of 3 of the requested asset type. As another example, the gene 427 represents a pediatric department at Facility 2. The department is located at an X coordinate 107.1 and a Y coordinate 50.9. There is a transfer cost of 10.6 and a utilization cost of 49.3 for the requested asset type. The department has an excess of 4 of the requested asset type. The X coordinates and Y coordinates may be used in calculating the distance from the donor department to the requesting department.

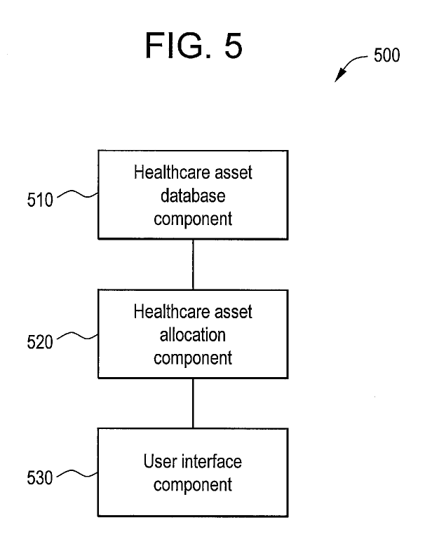

FIG. 5 illustrates a healthcare asset allocation system 500 in accordance with an embodiment of the present invention. The system 500 includes a healthcare asset database component 510, a healthcare asset allocation component 520, and a user interface component 530.

The healthcare asset allocation component 520 is in communication with the healthcare asset database component 510 and the user interface component 530.

In operation, the healthcare asset database component 510 stores healthcare asset data. The user interface component 530 receives a healthcare asset request. The healthcare asset allocation component 520 determines a healthcare asset allocation using a genetic algorithm, the healthcare asset data, genetic algorithm parameters, and/or the healthcare asset request. The user interface component 530 displays the healthcare asset allocation.

In certain embodiments, the healthcare asset allocation component 520 updates the healthcare asset data stored in the healthcare asset database component 510 with the healthcare asset allocation.

The healthcare database component 510 may be similar to the healthcare database component described above in reference to FIG. 1. The healthcare database component may comprise a database or a network of databases, which may be located at one or multiple locations. The healthcare asset data may be similar to the healthcare asset data described above in reference to FIGS. 1 and 2.

The healthcare asset allocation component 520 may be similar to the healthcare asset allocation component described above in reference to FIGS. 1 and 2.

The genetic algorithm parameters may be similar to the genetic algorithm parameters described above in reference to FIGS. 1, 2, and 3. The genetic algorithm parameters may include an approach type, a crossover rate, a crossover type, a mutation rate, a cost function, a population size, a selection method, a conversion criterion, and/or an end condition, for example. In certain embodiments, the crossover rate may be 95 percent, and the mutation rate may be 2 percent, for example. In certain embodiments, the cost function may include a distance between a donor department and a requesting department, a transfer cost, a utilization cost, and a facility transfer cost. In certain embodiments, the selection method may be a roulette wheel selection method.

The user interface component 530 may be similar to the user interface described above in reference to FIGS. 1 and 3. The user may input the healthcare asset request into the user interface component 530 as described above in reference to FIG. 1. The user interface component 530 may receive the request. The user may also set the genetic algorithm parameters using the user interface component 530 as described above in reference to FIG. 1.

The healthcare asset allocation component 520 may be implemented on a server or a network of servers, which may be located in one or multiple locations, for example. The healthcare asset allocation component 520 may comprise hardware, software, and/or firmware, for example. The healthcare asset allocation component 520 may determine the healthcare asset allocation using the method 100 described above in reference to FIG. 1. The healthcare asset allocation component 520 may receive a healthcare asset request through the user interface component 530. The healthcare asset allocation component 520 may create a plurality of potential solutions using the healthcare asset data and the genetic algorithm parameters. The healthcare asset allocation component 520 may apply a genetic algorithm to the plurality of potential solutions using the genetic algorithm parameters and the healthcare asset request to determine the healthcare asset allocation. The genetic algorithm may be similar to the genetic algorithm discussed above in reference to FIGS. 2 and 3.

The components, elements, and/or functionality of the interface(s) and system(s) described above may be implemented alone or in combination in various forms of hardware, firmware, and/or as a set of instructions in software, for example. Certain embodiments may be provided as a set of instructions residing on a computer-readable medium, such as a memory or hard disk, for execution on a general purpose computer or other processing device, such as, for example, one or more dedicated processors.

Thus, certain embodiments of the present invention provide a method for healthcare asset allocation. Further, certain embodiments of the present invention provide for a healthcare asset allocation system. Certain embodiments of the present invention use a genetic algorithm to determine a healthcare asset allocation. Certain embodiments provide a technical effect of determining a healthcare asset allocation using a genetic algorithm and healthcare asset data.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

Several embodiments are described above with reference to drawings. These drawings illustrate certain details of specific embodiments that implement the systems and methods and programs of the present invention. However, describing the invention with drawings should not be construed as imposing on the invention any limitations associated with features shown in the drawings. The present invention contemplates methods, systems and program products on any machine-readable media for accomplishing its operations. As noted above, the embodiments of the present invention may be implemented using an existing computer processor, or by a special purpose computer processor incorporated for this or another purpose or by a hardwired system.

As noted above, certain embodiments within the scope of the present invention include program products comprising machine-readable media for carrying or having machine-executable instructions or data structures stored thereon. Such machine-readable media can be any available media that can be accessed by a general purpose or special purpose computer or other machine with a processor. By way of example, such machine-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such a connection is properly termed a machine-readable medium. Combinations of the above are also included within the scope of machine-readable media. Machine-executable instructions comprise, for example, instructions and data which cause a general purpose computer, special purpose computer, or special purpose processing machines to perform a certain function or group of functions.

Certain embodiments of the invention are described in the general context of method steps which may be implemented in one embodiment by a program product including machine-executable instructions, such as program code, for example in the form of program modules executed by machines in networked environments. Generally, program modules include routines, programs, objects, components, data structures, etc., that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Certain embodiments of the present invention may be practiced in a networked environment using logical connections to one or more remote computers having processors. Logical connections may include a local area network (LAN) and a wide area network (WAN), which are presented here by way of example and not limitation. Such networking environments are commonplace in office-wide or enterprise-wide computer networks, intranets, and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules, and other data for the computer.

The foregoing description of embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principals of the invention and its practical application to enable one skilled in the art to utilize the invention in various embodiments and with various modifications as are suited to the particular use contemplated.

Certain features of the embodiments of the claimed subject matter have been illustrated as described herein; however, many modifications, substitutions, changes and equivalents will now occur to those skilled in the art. Additionally, while several functional blocks and relations between them have been described in detail, it is contemplated by those of skill in the art that several of the operations may be performed without the use of the others, or additional functions or relationships between functions may be established and still be in accordance with the claimed subject matter. It is, therefore, to be understood that the appended claims are intended to cover all such modifications and changes as fall within the true spirit of the embodiments of the claimed subject matter.

1. A healthcare asset allocation method, the method comprising:
receiving a healthcare asset request;
creating a plurality of potential solutions using healthcare asset data, wherein the healthcare asset data comprises data related to assets in a healthcare enterprise; and
determining a healthcare asset allocation by applying a genetic algorithm to the plurality of potential solutions using a plurality of genetic algorithm parameters and the healthcare asset request.

2. The method of claim 1, wherein the plurality of genetic algorithm parameters comprises at least one of an approach type, a crossover rate, a crossover type, a mutation rate, a cost function, a population size, a selection method, and an end condition.

3. The method of claim 1, wherein the step of applying the genetic algorithm comprises:
selecting a portion of the plurality of potential solutions using a cost function;
reproducing at least one new solution using the selected portion;
replacing the plurality of potential solutions with the at least one new solution and the selected portion; and
repeating the selecting, reproducing, and replacing steps until an end condition occurs.

4. The method of claim 3, wherein the reproducing step further comprises:
performing a crossover operation on the selected portion at a crossover rate using a crossover type; and
performing a mutation operation on the selected portion at a mutation rate.

5. The method of claim 4, wherein the crossover rate is 95 percent, and wherein the mutation rate is 2 percent.

6. The method of claim 3, wherein the cost function comprises a distance between a donor department and a requesting department, a transfer cost, a utilization cost, and a facility transfer cost.

7. The method of claim 3, wherein the cost function comprises Cₖ=Σ_{i=1,n}[(Xᵢ−Xₖ)²+(Yᵢ−Yₖ)²]^{1/2}+C_{T}+C_{U}+C_{Fik}.

8. The method of claim 1, wherein the creating step further comprises retrieving the healthcare asset data from a healthcare asset database component.

9. The method of claim 1, wherein the creating step further comprises specifying the genetic algorithm parameters.

10. The method of claim 1, wherein the plurality of potential solutions comprises 30 potential solutions.

11. The method of claim 1, wherein the healthcare asset data is stored in a healthcare asset database component.

12. The method of claim 1, wherein the method further comprises updating the healthcare asset data using the healthcare asset allocation.

13. An healthcare asset allocation system, the system comprising:
a healthcare asset database component adapted to store healthcare asset data; and
a healthcare asset allocation component adapted to determine a healthcare asset allocation using a genetic algorithm, the stored healthcare asset data, and a plurality of genetic algorithm parameters.

14. The system of claim 13, wherein the system further comprises a user interface component adapted to receive a healthcare asset request, wherein the healthcare asset allocation component is further adapted to use the received healthcare asset request to determine the healthcare asset allocation, and wherein the user interface component is further adapted to display the determined healthcare asset allocation.

15. The system of claim 13, wherein the healthcare asset allocation component is adapted to update the healthcare asset data with the healthcare asset allocation.

16. The system of claim 13, wherein the plurality of genetic algorithm parameters includes at least one of an approach type, a crossover rate, a crossover type, a mutation rate, a cost function, a population size, a selection method, and an end condition.

17. The system of claim 16, wherein the crossover rate is 95 percent, and wherein the mutation rate is 2 percent.

18. The system of claim 16, wherein the cost function comprises Cₖ=Σ_{i=1,n}[(Xᵢ−Xₖ)²+(Yᵢ−Yₖ)²]^{1/2}+C_{T}+C_{U}+C_{Fik}.

19. The system of claim 16, wherein the selection method is a roulette wheel selection method.

20. A computer-readable medium comprising a set of instructions for execution on a computer, the set of instructions comprising:
a data retrieval routine configured to retrieve healthcare asset data;
a user interface routine configured to receive a healthcare asset request; and
an asset allocation routine configured to determine a healthcare asset allocation using a genetic algorithm, a plurality of genetic algorithm parameters, the healthcare asset request, and the healthcare asset data.
